(19) United States
(12) Patent Application Publication
(10) Pub. No.: US 2004/0151731 A1
(43) Pub. Date: Aug. 5, 2004

(54) Methods and compositions involving T-lymphocyte reactivity with collagen in aortic tissue of abdominal aortic aneurysm patients

(76) Inventor: Douglas L. Jicha, Falmouth, ME (US)

Correspondence Address: KALOW & SPRINGUT LLP, 488 MADISON AVENUE, 19TH FLOOR, NEW YORK, NY, 10022, US

(21) Appl. No.: 10/357,851

(22) Filed: Feb. 4, 2003

(51) Int. Cl. A61K 38/00; A61K 31/525; A61K 31/573; A61K 38/13; C12N 5/08

(52) U.S. Cl. 424/185.1; 435/372; 514/11; 514/171; 514/251

(57) ABSTRACT

The present invention includes methods and compositions based on the discovery that T-lymphocytes from human AAA tissue have reactivity to the structural proteins in collagen.

This invention was made, in part, with government support from NCI CCSG Grant No. 5P30 CA 42014-09. The U.S. Government may have certain rights in this invention.

### BACKGROUND OF THE INVENTION

Abdominal aortic aneurysms involve dilation or stretching of the abdominal portion of the aorta. Typically, abdominal aortic aneurysms (AAA) are defined as arterial dilatations of greater than 1.5 times normal vessel diameter and are most common in the infrarenal aorta. AAAs can affect anyone, but it is most often seen in men 40 to 70 years old. In some cases, AAAs can rupture creating a life-threatening medical emergency where there is profuse bleeding into the abdominal cavity. Ruptured aneurysms occur more frequently in patients with larger (>5 cm) aneurysms. Approximately 15,000 people die each year of a ruptured abdominal aneurysm, making it the 13th leading cause of death in the United States. Mortality from rupture is estimated to be from 74 to 90 percent

AAAs are difficult to diagnose, approximately 75 percent of abdominal aortic aneurysms are asymptomatic and are detected during routine physical examination or during an unrelated radiologic or surgical procedure. Symptoms of abdominal aortic aneurysm may result from expansion or rupture of the aneurysm, pressure on adjacent structures, embolization or thrombosis. The most common symptom is abdominal, flank or back pain.

Several tests can-be used to diagnose an abdominal aortic aneurysm. These include a plain radiograph, B-mode ultrasound examination, computed tomographic (CT) scan, CT angiogram, magnetic resonance imaging (MRI) and angiography.

Treatment of AAAs depends on the size of the aneurysm, which has been correlated with the risk of rupture. For example, annual risk of rupture for an abdominal aneurysm from 5.0 to 5.9 cm in diameter is 5 percent, while the annual risk of rupture for an aneurysm 7 cm in diameter is 19 percent. Surgery is indicated in some cases to relieve pain, prevent rupture of the aneurysm and to prolong life. Emergency surgery is indicated for nearly all patients with known or suspected rupture because nonsurgical therapy of a ruptured abdominal aortic aneurysm is uniformly fatal. Unfortunately, surgery has unwanted complications such as infection, cardiac arrest, or death

The exact cause of AAA is unknown but risk factors include hypertension, infection, congenital weakening of the connective tissue component of the artery wall, trauma or atherosclerosis. Prior study of human AAA disease has documented a prominent inflammatory infiltrate in the media and adventitia. This inflammatory infiltrate pathologically distinguishes AAA disease from atherosclerotic occlusive disease and normal aorta.

The pathologic changes observed in AAA disease are further emphasized by the spectrum of AAA disease. Some clinicians first reported inflammatory aneurysms and described a subset of patients with retroperitoneal fibrotic changes and a white inflammatory aortic wall. Microscopic evaluation revealed a pan-inflammatory process that is CD3+ predominant. Some researchers have reported that 12% of patients with AAA disease that is not grossly of the inflammatory variety, manifest changes that are microscopically indistinguishable from inflammatory AAA disease.

If the T-lymphocytes present are acting in a directed pathologic process in AAA disease, several logical candidates for antigens are in the aortic wall. Extensive empirical description of the alterations in the aneurysmal aorta have demonstrated the importance of elastin and collagen in maintaining aortic wall integrity. Some clinicians report decreased elastin and collagen levels in the wall of AAAs. Other clinicians report increased to normal collagen levels in AAA disease.

Given the severity of AAA disease and the high risk of mortality, there is a need for assays, and effective therapies that can detect, prevent and treat AAA disease. Methods and compositions that lead to early diagnosis of AAAs would also be beneficial.

### SUMMARY OF THE INVENTION

It has been discovered that T-lymphocytes from human AAA tissue have reactivity to the key structural protein collagen. This reactivity is MHC class I restricted. Further, the necessary presenting cells are present in AAA tissue. With this discovery, methods, compositions and assays of the present invention allow rapid identification, prevention, diagnosis and treatment of patients suffering from AAA disease or rupture.

In one embodiment, the present invention provides an isolated or purified T-lymphocyte derived from abdominal aortic tissue, the T-lymphocyte is specifically reactive with collagen I, collagen III, fragment or combination thereof.

In another embodiment, the present invention provides a method of preventing or treating an abdominal aortic aneurysm or rupture in a mammal comprising administering to the mammal an effective amount of an immunosuppressive agent that inhibits T-lymphocyte reactivity with collagen I, collagen III, or epitope thereof, thereby preventing or treating the abdominal aortic aneurysm or rupture.

In yet another embodiment, the present invention provides a vaccine for preventing an abdominal aortic aneurysm or rupture in a mammal comprising an effective amount of collagen or epitope thereof isolated from abdominal aortic aneurysm tissue, the collagen capable of producing an immune response in a mammal.

In still another embodiment, the present invention provides a vaccine for preventing an abdominal aortic aneurysm or rupture in a mammal comprising an effective amount of collagen or epitope thereof isolated from abdominal aortic aneurysm tissue, the collagen capable of producing an immune response in a mammal.

In still yet another embodiment, the present invention provides a monoclonal antibody that specifically reacts with an epitope of collagen I, or collagen III, the epitope isolated from abdominal aortic aneurysm tissue.

In one exemplary embodiment, the present invention provides a kit for determining an individual's risk for developing and abdominal aortic aneurysm and/or rupture of AAA, comprising a container and a monoclonal antibody that specifically reacts with collagen I, collagen III, or epitope thereof, wherein the collagen I, collagen III or epitope thereof is synthesized or isolated from abdominal aortic aneurysm tissue.

In another exemplary embodiment, the present invention provides a method of stimulating or inhibiting an immune response in a mammal to prevent or treat an abdominal aortic aneurysm or rupture comprising administering to the mammal an effective amount of collagen I, collagen III, epitope or combination thereof so as to produce antibodies that inhibit T-lymphocyte reactivity with collagen I, collagen III or combination thereof, thereby stimulating the immune response to prevent or treat the abdominal aortic aneurysm or rupture.

In yet another exemplary embodiment, the present invention provides a method of stimulating or inhibiting an immune response in a mammal to prevent or treat an abdominal aortic aneurysm comprising administering to the mammal and effective amount of collagen I, collagen III, epitope or combinations thereof so as to bind T-lymphocytes, thereby preventing or treating the abdominal aortic aneurysm or rupture.

In still yet another exemplary embodiment, the present invention provides a method for determining a mammal's risk for developing an abdominal aortic aneurysm or rupture comprising: a) obtaining a sample of aortic tissue from the mammal; b) incubating the sample with one or more T-lymphocytes under suitable conditions so as to react the T-lymphocytes with collagen I, collagen III, epitope or combination thereof; and

c) determining the reactivity of the T-lymphocytes with collagen I, collagen III, or epitope or combination thereof by measuring cytokine release from the T lymphocytes derived from AAA tissue, wherein cytokine release or other T lymphocyte response indicates increased risk of developing an abdominal aortic aneurysm or rupture.

It is one aspect of the present invention to provide a pharmaceutical composition comprising an effective amount of an epitope of collagen I, collagen III, or combination thereof, wherein the epitope is isolated from abdominal aortic aneurysm tissue and is specifically reactive with T-lymphocytes.

It is another aspect of the present invention to provide a method of detecting collagen I, collagen III, epitope or combination thereof comprising: a) obtaining a sample of aortic tissue; b) incubating the sample with one or more T-lymphocytes under suitable conditions so as to react the T-lymphocytes with collagen I, collagen III, epitope thereof; or combination thereof and c) detecting collagen I, collagen III, epitope thereof or combination thereof by measuring proliferation, cytokine release or other reactivity of T-lymphocytes, wherein the response of the T-lymphocytes indicates the presence of collagen I, collagen III, epitope thereof, or combination thereof.

For a better understanding of the present invention together with other and further advantages and embodiments, reference is made to the following description taken in conjunction with the examples, the scope of which is set forth in the appended claims.

### BRIEF DESCRIPTION OF THE FIGURES

Preferred embodiments of the invention have been chosen for purposes of illustration and description, but are not intended in any way to restrict the scope of the invention. The preferred embodiments of certain aspects of the invention are shown in the accompanying figures, wherein:

FIG. 1 is a graphic illustration of cytokine release from AAA cells that had been cultured for 14 days. Cells were tested without any antigen (NoAg), with collagen I (Col I), collagen III (Col III) or elastin. In the middle group, autologous PBL (3000 rads) without long term cells are tested against the antigens. To the right, long term (responders) cells without PBL presenters are tested against antigens. In the left grouping, PBL presenters and long term cells are combined yielding marked collagen III reactivity.

FIG. 2 is a graphic illustration of interferon secretion from AAA cells. These cells were tested with autologous PBL or EBV (Epstein-Barr virus)-transformed B cells and no antigen (NoAg) or collagen I (Col I). EBV-transformed B cells were unable to act in antigen presentation.

FIG. 3 is a graphic illustration of interferon secretion from long term AAA cells tested with PBL presenting cells and no antibody or isotype matched MHC class I or class II blocking antibodies. Class I antibody blocking resulted in a 74% decrease in reactivity as demonstrated by interferon secretion.

### DETAILED DESCRIPTION OF THE INVENTION

The invention will now be described in connection with preferred embodiments. These embodiments are presented to aid in an understanding of the present invention and are not intended to, and should not be construed to, limit the invention in any way. All alternatives, modifications and equivalents that may become obvious to those of ordinary skill on reading the disclosure are included within the spirit and scope of the present invention.

This disclosure is not a primer on assays for preventing, diagnosing or treating AAA, basic concepts known to those skilled in the field of medical sciences and immunology have not been set forth in detail. Concepts such as choosing appropriate antigen, antibody reaction time and storage of AAA cells are readily determinable by those skilled in the industry and are generally described in the prior art. Attention is therefore directed to the appropriate texts and references known to those skilled in the art in regard to these matters.

The present invention is based on the discovery that T-lymphocytes from human AAA tissue have reactivity to the key structural protein collagen. This reactivity is MHC class I restricted. Further, the necessary presenting cells are present in AAA tissue.

Lymphocytes are white blood cells formed in the body's lymphoid tissue. The nucleus is round or ovoid with coarse, irregularly clumped chromatin while the cytoplasm is typically pale blue with azurophilic (if any) granules. Most lymphocytes can be classified as either T or B (with subpopulations of each); those with characteristics of neither major class are called null cells. T-lymphocyte are responsible for cell-mediated immunity. Two types have been identified—cytotoxic and helper T-lymphocytes. They are formed when lymphocytes circulate through the thymus gland and differentiate to thymocytes. When exposed to an antigen, they divide rapidly and produce large numbers of new T cells sensitized to that antigen. T-lymphocytes as used herein includes, but is not limited to, CD4, CD8, cytotoxic and helper T-lymphocytes.

The present invention provides isolated or purified T-lymphocyte derived from abdominal aortic tissue or peripheral blood in a patient with AAA disease, the T-lymphocyte is specifically reactive with collagen I, collagen III, fragment or combination thereof. Isolation of the T-lymphocyte can be accomplished using any method known in the art. Preferably, the T-lymphocyte may be isolated directly from cells, such as for example, abdominal aortic tissue, lymphatic cells, and the like. T-lymphocytes may be isolated from solutions of solubilized fraction by standard methods known in the art. Some suitable methods include gradient separation, antibody bead separation (positive and/or negatively selecting) and flow cytometric separation.

T-lymphocytes of the present invention can be purified by methods known in the art. In one embodiment of the present invention, culture methods were employed that were favorable to T lymphocyte proliferation. Further purification could be undertaken as listed above.

It has been discovered that the T-lymphocytes of the present invention are specifically reactive with collagen I or collagen III or fragment thereof. Collagen is a polypeptide substance comprising about one third of the total protein in mammalian organisms. It is the main constituent of large arteries, skin, connective tissue, and the organic substance of bones and teeth. Different forms of collagen are produced in the body but all consist of three alpha-polypeptide chains arranged in a triple helix. For example, collagen III is generally present in more distensible tissues in association with collagen I. Collagen is differentiated from other fibrous proteins, such as elastin, by the content of proline, hydroxyproline, and hydroxylysine; by the absence of tryptophan; and particularly by the high content of polar groups, which are responsible for its swelling properties.

Collagen I is composed of two α1(I) and one α2(1) chain, while Collagen III is composed of three α2(III) fragments. These are included in their precursor amino acid sequences (SEQ ID NO:1-3). Extensive post translational modification of these peptides includes cleavage of signal peptide, hydroxylation of selected prolines, addition of N-linked oligosaccharides, glycosylation of hydroxylysine residues, chain alignment and formation of disulfide bonds, triple-helical formation and completion of O-linked oligosaccharides. The homology of pro-collagen chains as judged by identical residues is 51% with α1(I) and α1(III) and 45% with α2(I) and α1(III).

Collagen I is an art recognized term and includes human collagen I that has two α1(I) chains (SEQ ID NO:1) and one α2(I) chain (SEQ ID NO: 2). Collagen III is an art recognized term and includes human collagen III that has three α2(III) fragments (SEQ ID NO:3).

T-lymphocytes may react with the entire protein of collagen I or collagen III. The protein may be the entire protein as it exists in nature, or an antigenic, preferably immunogenic, fragment of the whole collagen I or collagen III protein. Fragments include less than entire collagen I or collagen III sequence provided the fragment is antigenic and T-lymphocytes will specifically react with the fragment. These antigenic and/or immunogenic fragments of antigenic and/or immunogenic proteins may be identified by methods known in the art. Fragments containing antigenic sequences of collagen I or collagen III may be selected on the basis of generally accepted criteria of potential antigenicity and/or exposure. Such criteria include the hydrophilicity and relative antigenic index, as determined by surface exposure analysis of proteins. Amino acid domains predicted by these criteria to be surface exposed are selected preferentially over domains predicted to be more hydrophobic or hidden. Methods for isolating and identifying antigenic fragments from known antigenic proteins are well known in the art.

Collagen I, collagen III, fragment or combinations thereof, are epitopes for the T-lymphocytes. Epitopes include antigenic determinants recognized and bound by the T-cell receptor. Epitopes recognized by the T-cell receptor are often located in the inner, unexposed side of the antigen, and become accessible to the T-cell receptors after proteolytic processing of the antigen. Preferred epitopes of the present invention include collagen I or collagen III or fragment thereof derived from AAA tissue.

Surface sites on the T-lymphocytes of the present invention bind specifically with antigenic determinants on collagen I, collagen III or fragment thereof and induce an immune response. Typically, the immune response involves the release or inhibition of biologically active substances whose activities affect or play a role in the functioning of the immune system. The immune response is mediated by antigen-sensitized T-lymphocytes via lymphokines, cell to cell contact or direct cytotoxicity. This may take place in the absence of circulating antibody or where antibody plays a subordinate role. The immune response may include the release or inhibition of inflammatory mediators such cytokines and biologically active substances such as for example, interferon. The immune response may also include stimulation of colony stimulating factors, such as for example, GM-CSF (granulocyte-macrophage colony-stimulating factor) which stimulates the development of both granulocytes and macrophages in response to collagen I or collagen III or fragment thereof.

For purposes of the present invention, cytokines include non-antibody proteins secreted by inflammatory leukocytes such as T-lymphocytes and some non-leukocytic cells that act as intercellular mediators. They differ from classical hormones in that they are produced by a number of tissue or cell types rather than by specialized glands. They generally act locally in a paracrine or autocrine rather than endocrine manner. Interferons include proteins secreted by vertebrate cells such as for example T-lymphocytes, in response to a wide variety of inducers. They confer resistance against many different viruses, inhibit proliferation of normal and malignant cells, impede multiplication of intracellular parasites, enhance macrophage and granulocyte phagocytosis, augment natural killer cell activity, and show several other immunomodulatory functions. Preferred interferons include gamma interferon A type II interferon secreted by T-lymphocytes.

T-lymphocytes of the present invention are reactive with collagen I or collagen III or fragments thereof located in AAA tissue and PBL are reactive with collagen I or collagen III or fragments thereof. Thus with this discovery, methods of diagnosing, preventing or treating AAA or rupture can be employed. It has been found that cytokines, and/or interferons are released by the T-lymphocytes and contribute to the inflammatory process in AAA disease.

Abdominal aortic aneurysm or AAA disease includes an aneurysm in the part of the aorta continuing from the thoracic region and giving rise to the inferior phrenic, lumbar, median sacral, mesenteric, iliac, renal, and ovarian or testicular arteries. Typically, aortic aneurysm include a sac formed by the dilatation of the wall of the aorta. In severe cases the AAA can rupture or tear the aortic tissue creating a life-threatening medical emergency where there is profuse bleeding into the abdominal cavity. Ruptured aneurysms occur more frequently in patients with larger (>5 cm) aneurysms. This method may also diagnose and/or treat aneurysms in other locations including iliac, femoral, thoracic and peripheral vessels.

In one embodiment, the present invention can be used to diagnose a mammal with AAA disease or rupture or be used to determining the mammal's risk for developing an abdominal aortic aneurysm or rupture comprising: obtaining a sample of T-lymphocytes from AAA tissue of the mammal; and incubating the sample under suitable conditions so as to react the T-lymphocytes with collagen I, collagen III, or epitope thereof, and determining the reactivity of the T-lymphocytes with collagen I, collagen III, or epitope thereof by measuring cytokine release or T-lymphocyte proliferations, wherein cytokine release indicates increased risk of developing an abdominal aortic aneurysm or rupture.

Suitable conditions for incubation are readily determinable by those skilled in the art of immunology and include appropriate cell culture, growth media, temperature and the like. In any event, the present invention is not limited to any one particular incubation condition.

The present invention can be used to diagnosis AAA disease or rupture risk. Diagnosis includes the determination of the nature of AAA disease or rupture. Risk for developing AAA disease includes assessing an individual's propensity for AAA disease or rupture.

With the recognition that T-lymphocytes react with collagen, collagen I, collagen III, or fragment thereof, diagnostic assays can be made. Assays for detecting T-lymphocyte activity or collagen activity can follow known formats such as for example, such as standard blot and ELISA formats. These formats are normally based on incubating an antibody with a sample suspected of containing the T-lymphocyte or collagen and detecting the presence of a complex between the antibody and the protein (i.e., T-lymphocyte or collagen). The antibody is labeled either before, during, or after the incubation step. The protein is preferably immobilized prior to detection. Immobilization may be accomplished by directly binding the protein to a solid surface, such as a microtiter well, or by binding the protein to immobilized antibodies. In a preferred embodiment, a protein is immobilized on a solid support through an immobilized first antibody specific for the protein. The immobilized first antibody is incubated with a sample suspected of containing the protein. If present, the protein binds to the first antibody. A second antibody, also specific for the protein, binds to the immobilized protein. The second antibody may be labeled by methods known in the art. Non-immobilized materials are washed away, and the presence of immobilized label indicates the presence of the protein. These types of assays can also be used to measure increases or decreases in cytokine, interferon and or T-lymphocyte proliferation.

With the recognition that T-lymphocytes react with collagen, collagen I, collagen III, or fragment thereof, assays for detecting T-lymphocyte activity or collagen activity can follow FACS formats. These formats are normally used to detect cell surface antigens. Briefly, the protein of interest (for example protein on CD4, CD8, collagen) on an intact whole cell is recognized by a fluorescent labeled antibody, then flows thru a detection machine that counts the labeled cells.

Assays that detect cytokine or GM CSF release can be employed using known formats. In one embodiment of the present invention, T-lymphocyte cells are grown in culture, and then exposed to various antigens such as collagen (i.e., collagen I, II, III or fragment or combination thereof). The supernatant fluid from the growing cells contains released cytokines (i.e., interferon) or colony stimulating factor (i.e., GM CSF). These released substances are detected by standard ELISA, where, for example, wells are coated with antibody specific to interferon or GM CSF, supernatants suspected of containing interferon or GM CSF are incubated on the wells, then washed and reacted with biotin-labeled anti-IFN or GM CSF antibodies. The level of the label detected reflects the level of the cytokine or GM CSF in the cell supernatant, and the level of cytokine or GM CSF made by the cells.

The present invention is not limited to any particular label. Labels useful for the present invention include radioactive and non-radioactive labels known in the art. Some examples of useful radioactive labels include ³²P, ¹²⁵I, ¹³¹I, ³⁵S, ¹⁴C, and ³H. Some examples of non-radioactive labels include enzymes, chromophores, atoms and molecules detectable by electron microscopy, and metal ions detectable by their magnetic properties. Some useful enzymatic labels include enzymes that cause a detectable change in a substrate. Some useful enzymes and their substrates include, for example, horseradish peroxidase (pyrogallol and o-phenylenediamine), beta-galactosidase (fluorescein beta-D-galactopyranoside), and alkaline phosphatase (5-bromo-4-chloro-3-indolyl phosphate/nitro blue tetrazolium), and the like. Useful chromophores include, for example, fluorescent, chemiluminescent, and bioluminescent molecules, as well as dyes.

Some specific chromophores useful in the present invention include, for example, fluorescein, rhodamine, Texas red, phycoerythrin, umbelliferone, luminol. As used in the present invention, the term “detecting” refers to identification of a detectable moiety or label that may be on the protein by methods known in the art. Some suitable detection methods include the ability to identify a moiety by electromagnetic characteristics, such as, for example, charge, light, fluorescence, chemiluminescense, changes in electromagnetic characteristics such as, for example, fluorescence polarization, light polarization, dichroism, light scattering, changes in refractive index, reflection, infrared, ultraviolet, and visible spectra, and all manner of detection technologies dependent upon electromagnetic radiation.

The protein collagen I, collagen III or fragment thereof has been found, unexpectedly, to be an antigenic for T-lymphocytes. Thus, collagen I, collagen III or fragments thereof, can be used to vaccinate or protect the mammal from AAA disease or rupture. Vaccines include antigenic proteins of collagen I, collagen III, or fragments thereof preferably derived from AAA, or synthetically constructed, that are administered for the prevention, amelioration, or treatment of AAA. Disease is significantly inhibited in vivo if the inhibition is sufficient to prevent or reduce the symptoms of AAA in a mammal. Tolerizing as used herein includes the specific lack of immune responsiveness to the antigen, in the present case, collagen, collagen I, collagen III or fragment thereof as a result of prior contact with the collagen. Thus, the individual can develop tolerance to the AAA disease by administering repeated doses of, collagen, collagen I, collagen III, fragment or combination thereof. The term “tolerizer” includes a tolerizing dose of collagen, collagen I, collagen III or fragment thereof.

Vaccines comprising the collagen, collagen I, collagen III, or functional analogs thereof may be used to prevent or inhibit rupture or AAA i.e., dilatation of the wall of the aorta in accordance with the invention. Functional analogs of the collagen protein for this purpose include fragments and substitution, addition or deletion that produce an immune response in a mammal. To be useful, the vaccine is non-toxic to the mammal being immunized. If the vaccine is toxic, it may be detoxified by methods known in the art. Such methods include, for example, providing antigenic, non-toxic fragments of the entire protein or detoxifying a protein by, for example, binding the toxin to a carrier molecule that destroys toxicity, but does not affect antigenicity. The carrier molecule is typically another protein. The vaccine may be the full length protein or a fragment thereof. The length of the fragment is not critical as long as the fragment is immunogenic and non-toxic. Therefore, the fragment should contain sufficient amino acid residues to define the epitope.

The present invention further includes vaccine compositions for immunizing mammals, including humans, to prevent AAA or rupture by administering an effective amount of collagen, collagen I, collagen III or epitope thereof isolated from abdominal aortic aneurysm tissue, the collagen is capable of producing an immune response in the mammal. Vaccine compositions include an immunogenic antigen i.e., collagen I or collagen III or fragment thereof as described above in a suitable carrier. The vaccine may include adjuvants, such as muramyl peptides, and lymphokines, such as, interleukin-1 and interleukin-6. The antigen may be adsorbed on suitable particles, such as aluminum oxide particles, or encapsulated in liposomes, as is known in the art.

The present invention includes administering an effective amount of collagen, collagen I, collagen III or fragment thereof. As used herein, an effective amount is that amount effective to achieve the specified result of preventing or treating AAA disease or rupture. Preferably, AAA or rupture is prevented or treated without serious side effects.

The maximal dosage for preventing or treating a mammal having AAA is the highest dosage that does not cause undesirable or intolerable side effects. Minimal dosage is the lowest dosage where efficacy is first observed. In accordance with the present invention, the dosage unit can be in any form. As will be apparent to the skilled artisan, the concentration of the collagen, collagen I, collagen III or fragment will vary with the choice of administration to the mammal. For example, if the collagen, collagen I, collagen III or fragment thereof is administered by injection to the mammal, the dosage unit is a syringe containing an effective amount of the collagen. An effective amount of the collagen, collagen I, collagen III or fragment thereof for systemic administration can range from about 0.01 mg/kg to 50 mg/kg administered once or twice per day. However, different dosing schedules can be utilized depending on (i) the potency of an individual collagen at inhibiting AAA or rupture, (ii) the severity or extent of the pathological disease state, or (iii) the pharmacokinetic behavior of a given collagen. In any event, the practitioner is guided by skill and knowledge in the field, and the present invention includes without limitation dosages, which are effective to achieve the described effect.

The method of the present invention includes administering a pharmaceutical composition comprising collagen, collagen I, collagen III or fragment in an amount which is effective for preventing or treating AAA or rupture. Pharmaceutical compositions include the collagen, collagen I, collagen III or fragment in a suitable dosage form such as for example, injection, oral formulations such as tablets, capsules, pills, troches, elixirs, suspensions, syrups, wafers, chewing gum and the like can be employed to provide the desired amount of collagen, collagen I, collagen III or fragment. Alternatively, delivery of the collagen includes topical application, such as gels, salves, lotions, creams ointments and the like.

The methods of the present also include preventing or treating an abdominal aortic aneurysm or rupture in a mammal comprising administering to the mammal an effective amount of an immunosuppressive agent that inhibits T-lymphocyte reactivity with collagen I or collagen III or epitope thereof, thereby preventing or treating the abdominal aortic aneurysm or rupture. Suitable immunosuppressive agents will inhibit T-lymphocytes from releasing cytokines, interferons and undesirable biologically active substance that contribute to AAA or rupture. Immunosuppressive agents include, but are not limited to, azathioprine, cyclosporine, methotrexate, prednisone, methylprednisolone, prednisolone, hydrocortisone, cortisone and combinations thereof. These immunosuppressive agents can be administered in different routes and dosage forms as discussed above.

Other methods for the prevention or treatment of AAA or rupture include administering antibodies that are raised against and block epitopes of collagen, collagen I or collagen III or fragments thereof. An “antibody” in accordance with the present specification is defined broadly as a protein that binds specifically to an epitope. The antibody may be polyclonal or monoclonal. Antibodies further include recombinant polyclonal or monoclonal Fab fragments prepared in accordance with the method known in the art. Polyclonal antibodies are isolated from mammals that have been innoculated with the protein or a functional analog in accordance with methods known in the art. Briefly, polyclonal antibodies may produced by injecting a host mammal, such as a rabbit, mouse, rat, or goat, with the protein or a fragment thereof capable of producing antibodies that distinguish between mutant and wild-type protein. The peptide or peptide fragment injected may contain the wild-type sequence or the mutant sequence. Sera from the mammal are extracted and screened to obtain polyclonal antibodies that are specific to the peptide or peptide fragment.

Preferred antibodies have reactivity against collagen, collagen I, collagen III or epitope thereof. The antibodies are preferably monoclonal. Monoclonal antibodies may be produced by methods known in the art. For example, in order to produce monoclonal antibodies, a host mammal is inoculated with a peptide or peptide fragment as described above, and then boosted. Spleens are collected from inoculated mammals a few days after the final boost. Cell suspensions from the spleens are fused with a tumor cell and monoclonal antibodies are generated from the fused cells.

The present invention includes kits for determining an individual's risk for developing an abdominal aortic aneurysm or rupture, comprising a container and a monoclonal antibody that specifically reacts with AAA tissue T-lymphocyte or collagen, collagen I, collagen III or fragment thereof. The kit may include commercially prepared reagent sets, with accessory devices, containing all of the major components and literature necessary to perform one or more designated diagnostic tests or procedures.

They may be for laboratory or personal use.

Having now generally described the invention, the same may be more readily understood through the following reference to the following examples, which are provided by way of illustration and are not intended to limit the present invention unless specified.

### EXAMPLES

The examples below demonstrate that lymphocytes from human AAA tissue have reactivity to the key structural protein collagen. This reactivity is MHC class I restricted.

Further, the necessary presenting cells are present in AAA tissue. This suggests an important pathologic role for T lymphocytes in AAA disease that is different from T lymphocytes in atherosclerosis.

### Example 1

### T-lymphocytes from Human AAA Tissue Have Reactivity to Collagen

Patient Demographics: Ten human abdominal aortic aneurysms (AAAs) were studied.

The mean AAA size was 7 cm (range 3.5 to 10 cm) and four of the 10 specimens were form patients with ruptured AAAs. Two female and eight male patients had a mean age 25 of 70 years with a range of 61 to 78 years. Atherosclerotic risk factors in the group included hypertension in three patients, tobacco use in six patients, hypercholesterolemia in one patient and no patient was a known diabetic. All specimens were obtained in accordance with University of Utah IRB.

Normal (NL) aortic tissue, which served as control tissue, was obtained from three patients ranging in age from 18 to 52 years. In two cases, tissue was obtained from brain dead organ donors suffering from trauma. In one additional case, brain death was from rupture of an intracranial aneurysm (NL1) with grossly normal aortic tissue. Normal peripheral blood lymphocytes (PBL) were obtained from normal volunteers, with no family history of connective tissue disorders, ranging in age from 18 to 52 years.

Aortic Tissue Preparation: AAA tissue and normal aortic tissue was minced into 1 to 5 mm fragments and subjected to an 18 to 24 hour triple enzyme digest using 4 mg of deoxyribonuclease, 40 mg of collagenase and 100U of hyaluronidase (Sigma, St. Louis, Mo.). Digests were filtered through a nylon mesh and washed in Hank's balanced salt solution (HBSS). “Long Term” cultures were initiated on 24-well plates (Costar, Cambridge, Mass.) coated with OKT-3 using 0.05 M carbonate-bicarbonate coating buffer, pH 9.6 (Sigma). Cells were grown in complete media consisting of RPMI-1640, 10% fetal calf serum, antibiotics, 20 mM HEPES buffer,

2 mM L-glutamine and 200U/ml interleukin-2. Cultures were counted (with trypan blue exclusion), split and/or fed at least every four days and were initially left on OKT-3 coated plates for 48 to 72 hours.

FACS Analysis: Cell surface antigens were detected employing a FACS can (Becton Dickinson, Mountain View, Calif.). FACS analysis was subsidized in part as a core facility at the University of Utah via NCI CCSG Grant #5P30 CA 42014-09. Staining was performed on single cell suspensions in a FACS buffer containing 5% heat inactivated fetal bovine serum and 0.1% sodium azide in HBSS without phenol red (Biofluids, Rockville, Md.) at 4° C. with appropriately titered monoclonal antibodies for 45 minutes. Anti-leu 3 (CD4), anti-leu 2 (CD8) and isotype control IgG-FITC antibodies were obtained from Becton Dickinson.

Cytokine Release Assays: Cells from fresh digest (“Bulk”) or from cultures of 12 to 14 days (“Long Term”) were harvested and plated in 96-well flat bottomed plates (Costar) in complete medium. In all cases, 2×10⁶ responder cells were plated in 0.2 ml total volume. Human collagen I, human collagen III (Sigma) and human elastin (Elastin Products, Inc., Owensville, Mo.) were added to a final concentration of 1 mg/ml with polymyxin B at 10 μg/ml (Sigma). CD-3 was employed as a positive control. For Long Term culture assays, 2×10⁶ feeders were added of PBL, BULK, or EBV-transformed cells previously irradiated with 3000 rads. PBL were prepared from whole blood with Lympholyte Separation Medium (Organon Teknika Corp., Durham, N.C.). EBV-transformed B cell lines were established using standard techniques with the assistance of the University of Utah Clinical Research Center. In all cases, supernatants were harvested after a 24 hour incubation at 37° C.

In antibody blocking assays, isotype matched preservative free monoclonal antibodies IQU9 (anti-HLA-DR, DP, DQ) and W6/32 (anti-HLA-A, B, C) (Biodesign International, Kennebunk, Me.) at 2.5 μg/ml were cultured with presenting PBL for one half hour and PBL plus Long Term cells for an additional half hour prior to the addition of antigen. FACS analysis confirmed the saturation of PBL receptors with antibodies at this concentration.

Interferon (γIFN) and GM-CSF ELISAs: 96 well flat bottom Corning plates were coated with 2 μg/ml of mouse anti-human γIFN or rat anti-human GM-CSF antibodies (Pharmingen, San Diego, Calif.) in 50 mM tris pH 9.5 at 4° C. overnight. Plates were then washed with 0.05% Tween 20 in PBS and blocked for two hours. After washing, plates were coated with samples and standards of recombinant γIFN and GM-CSF (Pharmigen).

After incubation, plates were washed and incubated for forty-five minutes with biotinylated mouse anti-human γIFN or rat anti-human GM-CSF antibodies (Pharmigen).

Next, plates were washed and incubated for thirty minutes with HRP-avidin D (Vector, Burlingame, Calif.) and developed with ABTS. Plates were read at 405 nm. Standard curves and data points were analyzed using DeltaSoft (Biometallic, Inc. version 1.80).

Statistical Analysis: All cytokine secretion values are presented ± estimated standard error of the mean (±est. SEM) as calculated by DeltaSoft. Positive levels for cytokine secretion of 500 ρg/ml of γIFN or of GM-CSF above the maximum estimated standard error of the mean were employed in all cases. In all Tables presented, basal cytokine secretion with no antigen was subtracted as background in BULK and Long Term assays. Additionally, in Long Term assays, controls of feeders (PBL) with antigen were also subtracted as background.

Results for Example 1

Fresh digests from AAA tissue (BULK cells) and normal PBL controls were cultured with OKT-3 coated plates and interleukin-2. As shown in Table #1, significant T cell enrichment was obtained during the 12 to 14 day culture period. While absolute numbers of cells from AAA specimens increased from between 0.3 and 5.8 fold (mean 1.9), T lymphocytes expanded from 0.33 to 133 fold (mean 2.8). In Long Term cultures, CD4+ cells ranged from 19 to 41% and CD8+ cells from 4 to 76%. Normal aortic tissue expanded more slowly than BULK AAA cells and morphologic evidence of fibroblasts were present upon culture inspection.

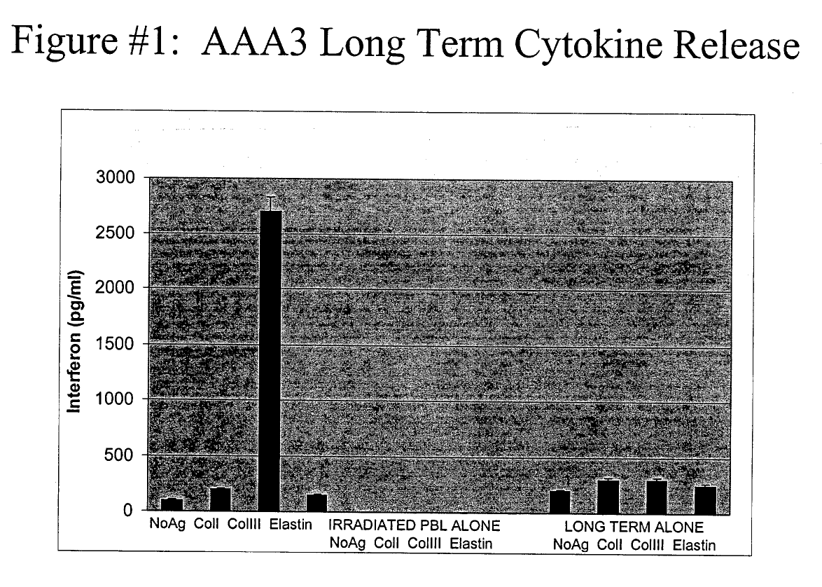

Long Term AAA cells were tested for their reactivity to aortic structural proteins in cytokine release assays. A positive assay is shown in FIG. 1, where exposure to collagen III resulted in 2578±206 ρg/ml of γIFN secretion. Controls of autologous irradiated PBL alone and Long Term cells alone exposed to collagen I, collagen III and elastin did not result in significant γIFN secretion. Antigen presenting cells were necessary for specific cytokine secretion.

All Long Term cultures that were tested in cytokine release assays are shown in Tables #2 and 3. Due to a limited supply of autologous PBL presenting cells, limited testing (with collagen I only) was possible in AAA5 and AAA9. Of the remaining eight Long Term cultures that were more fully tested, five were positive for specific cytokine exposure to collagen I or III. In three cases, cytokine secretion was present for both collagen I and III. In no cases was elastin reactive by cytokine secretion in these assays. Control PBL Long Term assays are also shown and were not reactive in any case with only low level cytokine secretion.

To rule out in vitro stimulation during triple enzyme digest as a mechanism for the observed reactivity, an additional AAA specimen was mechanically dispersed and a single cell suspension was obtained without a triple enzyme digest. Subsequent test of Long Term cells yielded γIFN secretion of 1212±427 ρgml with collagen 1 and 1199±420 ρg/ml with collagen III. Reactive cells were obtained with immediate mechanical dispersion and subsequent culture.

To further evaluate the functional nature of T lymphocytes in AAA tissue, BULK AAA cells, normal PBL and normal aortic tissue cellular infiltrates were tested for their reactivity to the aortic structural proteins collagen I, collagen III, and elastin in cytokine release assays. Significant cytokine secretion was observed in two cases. BULK AAA10 cells secreted 731±129 ρg/ml of γIFN with exposure to collagen III and BULK AAA3 secreted 428±93 ρg/ml of GM-CSF with exposure to collagen I. In no case did elastin result in significant cytokine release. Control of normal PBL (n=2) and of normal aorta (n=2) were not reactive.

To further investigate the relevance of the cytokine secretion observed in Long Term cultures, BULK cells from the autologous AAA tissue were irradiated and employed as presenting cells in the place of PBL. These assays were performed at the same time as the assays in Tables # 2 and 3. Results are shown in Table #4. As predicted by the PBL presenting assays, cytokine stimulation was not present with BULK presenting cells in AAA6 and AAA9. AAA2 with BULK presentation did produce 1212±427 ρg/ml of γIFN with exposure to collagen I which is less than the 4474±798 ρg/ml of γIFN with PBL presenting cells. AAA2 with BULK presentation did not result in significant secretion with collagen III exposure. AAA1 cells were not reactive. Cells present in AAA tissue were capable of presenting collagen to LONG TERM cells, but appeared less efficient than PBL presenting cells.

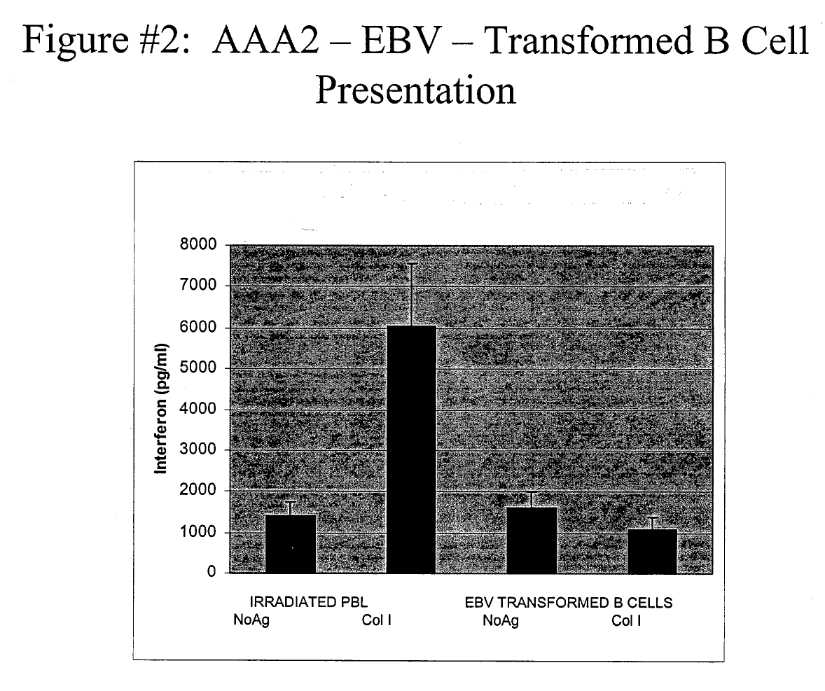

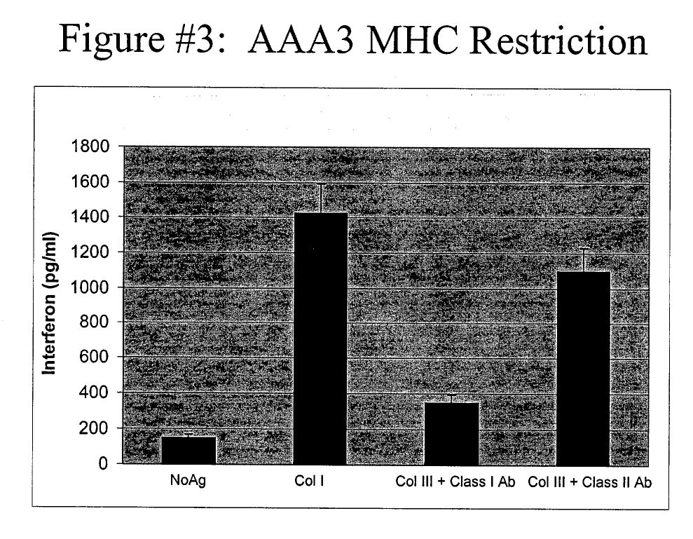

The nature of antigen presentation with Long Term cells was further investigated with EBV-transformed autologous B cells. As shown in FIG. 2, PBL were able to elicit significant γIFN secretion with collagen I exposure but, in the same assay, EBV-transformed B cells did not effectively present collagen I. PBL presentation was further evaluated by employing isotype matched MHC class I and class II antibodies. As shown in FIG. 3, class I blocking resulted in a 74% reduction in γIFN secretion while class II blocking resulted in a 23% reduction.

Discussion of Example 1

In this report, it is shown that T lymphocytes from AAA are reactive to collagen I and III. Presenting cells are necessary for this T lymphocyte reactivity which is class I restrictive. Further, the necessary presenting cells are present in AAA tissue. We believe that this work outlines a significant destructive role for T lymphocytes in AAA disease.

Collagen I and III are important structural proteins in human abdominal aortic tissue. Some researchers suggest that collagen is important in the burst strength of the aorta and therefore may be more important in aortic rupture than elastin which has been associated with dilatation. Four ruptured AAA specimens were included in the current study. It may be noteworthy that one of the two patients with positive BULK cytokine secretion was a ruptured AAA. Further, in the Long Term cytokine secretion assays, the highest levels of γIFN and GM-CSF were noted in another AAA rupture specimen. This may suggest that T lymphocyte reactivity is one of the causes leading to aneurysm rupture.

The cross reactivity observed with collagen I and III is not unexpected. Both collagen I and III are group 1 collagen molecules with a significant homology. The homology of procollagen chains as judged by identical residues is 51% with α1 (1) and α1 (III) and 45% with α2 (I) and α1 (III). Further, T cell recognition of collagen II has been shown to involve recognition of carbohydrates bound to the collagen. Similar post-translational modification of collagen I and III could be important in their recognition by AAA derived cells.

The role of T lymphocytes in atherosclerosis appears to be different than that in AAA disease. Some researchers have reported that CD4+ T cell clones derived from atherosclerosis plaques have increased proliferation with exposure to oxidized low density lipoprotein (oxLDL). We have not tested our polyclonal populations from AAA for reactivity to oxLDL, but, our results taken with this prior report suggests an alternative role for T lymphocytes in AAA disease, that may be central initiation and propagation of aneurismal disease.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice thin the art to which the invention pertains and as may be applied to the essential features herein before set forth and as follows.

TABLE #1
Long Term Culture CD4 and CD8 T Lymphocyte Analysis

| | | BULK | | # of Days | Long Term | |
|---|---|---|---|---|---|---|
| | Specimen # | % CD4 | % CD8 | in Culture | % CD4 | % CD8 |
| AAA | AAA1 | 5 | 15 | 13 | 19 | 76 |
| | AAA2 | 19 | 13 | 14 | 24 | 65 |
| | AAA3 | 23 | 5 | 14 | 30 | 44 |
| | AAA4 | 1 | 2 | 14 | 30 | 53 |
| | AAA5 | <1 | <1 | 13 | 24 | 4 |
| | AAA6 | 24 | 7 | 13 | 25 | 52 |
| | AAA7 | ND | ND | 13 | ND | ND |
| | AAA8 | <1 | 7 | 13 | 41 | 21 |
| | AAA9 | ND | ND | 13 | ND | ND |
| | AAA10 | 1 | 3 | 13 | 26 | 66 |
| Normal | NL 1 | <1 | 2 | 10 | ND | ND |
| Aorta | NL 2 | <1 | 2 | 14 | 10 | 15 |
| | NL 3 | ND | ND | 13 | ND | ND |
| PBL | PBL 1 | ND | ND | 12 | ND | ND |
| | PBL 2 | ND | ND | 12 | ND | ND |
| | PBL 3 | ND | ND | 14 | 31 | 46 |

ND = not done, FACS analysis on normal PBL revealed 31% CD4, 15% CD8 and <1% IgG-FITC control

TABLE #2
Long Term Cultures - _{γ}Interferon

| | | ANTIGEN | |
|---|---|---|---|
| Specimen # | Collagen I | Collagen III | Elastin |
| AAA | | | |
| AAA1 | 621 ± 150 | 942 ± 164 | 29 ± 107 |
| AAA2 | 4474 ± 798 | 809 ± 554 | ND |
| AAA3 | 93 ± 113 | 2578 ± 206 | 16 ± 80 |
| AAA4 | 0 ± 0 | 0 ± 0 | 0 ± 0 |
| AAA5 | 0 ± 241 | ND | ND |
| AAA6 | 676 ± 204 | 647 ± 210 | ND |
| AAA7 | 2386 ± 261 | 2318 ± 248 | 283 ± 337 |
| AAA8 | 790 ± 66 | 551 ± 62 | 100 ± 47 |
| AAA9 | 184 ± 114 | ND | ND |
| AAA10 | 62 ± 64 | 78 ± 69 | 0 ± 83 |
| PBL | | | |
| PBL1 | 208 ± 96 | 140 ± 89 | 22 ± 73 |
| PBL2 | 274 ± 131 | ND | ND |
| PBL3 | 0 ± 0 | 162 ± 36 | 0 ± 0 |

All values are in ρg/ml ± est. SEM, ND = not done

TABLE #3
Long Term Cultures Cytokine Secretion - GM-CSF

| | | ANTIGEN | |
|---|---|---|---|
| Specimen # | Collagen I | Collagen III | Elastin |
| AAA | | | |
| AAA1 | 1000 ± 136 | 432 ± 116 | 199 ± 112 |
| AAA2 | 734 ± 192 | 1129 ± 204 | ND |
| AAA3 | 0 ± 75 | 0 ± 79 | 0 ± 73 |
| AAA4 | 0 ± 0 | 0 ± 0 | 0 ± 167 |
| AAA5 | 197 ± 210 | ND | ND |
| AAA6 | 355 ± 133 | 355 ± 139 | ND |
| AAA7 | 494 ± 245 | 507 ± 233 | 351 ± 251 |
| AAA8 | 0 ± 54 | 0 ± 56 | 0 ± 57 |
| AAA9 | 134 ± 121 | ND | ND |
| AAA10 | 109 ± 43 | 108 ± 43 | 4 ± 28 |
| PBL | | | |
| PBL1 | 0 ± 99 | 0 ± 93 | 0 ± 76 |
| PBL2 | 0 ± 153 | ND | ND |
| PBL3 | 75 ± 92 | 0 ± 95 | 111 ± 92 |

All values are in ρg/ml ± est. SEM, ND = not done

TABLE #4
Long Term Culture _{γ}Interferon and GM-CSF Cytokine Secretion
with BULK presenting cells

| | | | | |
|---|---|---|---|---|
| | | | ANTIGEN | |
| Cytokine | Specimen # | Collagen I | Collagen III | Elastin |

| | | | | |
|---|---|---|---|---|
| _{γ}Interferon | AAA1 | 0 ± 104 | 0 ± 108 | 0 ± 111 |
| | AAA2 | 2295 ± 592 | 294 ± 457 | 45 ± 421 |
| | AAA6 | 5 ± 119 | 393 ± 174 | ND |
| | AAA9 | 68 ± 103 | 115 ± 112 | 36 ± 103 |
| GM-CSF | AAA1 | 312 ± 163 | 0 ± 158 | 0 ± 132 |
| | AAA2 | 0 ± 210 | 0 ± 207 | 0 ± 224 |
| | AAA6 | 44 ± 146 | 421 ± 173 | ND |
| | AAA9 | 0 ± 163 | 0 ± 160 | 0 ± 167 |

All values are in ρg/ml ± est. SEM, ND = not done

What is claimed is:

1. An isolated or purified T-lymphocyte derived from abdominal aortic tissue, the T-lymphocyte is specifically reactive with collagen I or collagen III or fragment thereof.

2. A method of preventing or treating an abdominal aortic aneurysm or rupture in a mammal comprising administering to the mammal an effective amount of an immunosuppressive agent that inhibits T-lymphocyte reactivity with collagen I or collagen III or epitope thereof, thereby preventing or treating the abdominal aortic aneurysm or rupture.

3. A method according to claim 2, wherein the immunosuppressive agent is selected from the group such as azathioprine, cyclosporine, methotrexate, prednisone, methylprednisolone, prednisolone, hydrocortisone, cortisone and combinations thereof.

4. A vaccine for preventing an abdominal aortic aneurysm or rupture in a mammal comprising an effective amount of collagen or epitope thereof isolated from abdominal aortic aneurysm tissue, the collagen capable of producing an immune response in a mammal.

5. A vaccine according to claim 4, wherein the immune response is production of antibodies specifically reactive with collagen I, or collagen III or epitope thereof.

6. A vaccine according to claim 4, wherein the immune response is the production of cytokines.

7. A monoclonal antibody that specifically reacts with an epitope of collagen I, or collagen III, the epitope isolated from abdominal aortic aneurysm tissue.

8. A kit for determining an individual's risk for developing and abdominal aortic aneurysm and/or rupture of AAA, comprising a container and a monoclonal antibody that specifically reacts with collagen I, collagen III, or epitope thereof, wherein the collagen I, collagen III or epitope thereof is synthesized or isolated from abdominal aortic aneurysm tissue.

9. A method of stimulating or inhibiting an immune response in a mammal to prevent or treat an abdominal aortic aneurysm or rupture comprising administering to the mammal an effective amount of collagen I, collagen III, epitope thereof or combination thereof so as to produce antibodies that inhibit T-lymphocyte reactivity with collagen I, collagen III or combination thereof, thereby stimulating the immune response to prevent or treat the abdominal aortic aneurysm or rupture.

10. A method of stimulating or inhibiting an immune response in a mammal to prevent or treat an abdominal aortic aneurysm comprising administering to the mammal and effective amount of collagen I, collagen III, epitope thereof or combinations thereof so as to bind T-lymphocytes, thereby preventing or treating the abdominal aortic aneurysm or rupture.

11. A method for determining a mammal's risk for developing an abdominal aortic aneurysm or rupture comprising
(a) obtaining a sample of aortic tissue from the mammal;
(b) incubating the sample with one or more T-lymphocytes under suitable conditions so as to react the T-lymphocytes with collagen I, collagen III, epitope thereof or combination thereof, and
(c) determining the reactivity of the T-lymphocytes with collagen I, collagen III, or epitope thereof or combination thereof by measuring cytokine release from the T lymphocytes derived from AAA tissue, wherein cytokine release or other T lymphocyte response indicates increased risk of developing an abdominal aortic aneurysm or rupture.

12. A pharmaceutical composition comprising an effective amount of an epitope of collagen I, collagen III, or epitope thereof, wherein the epitope is isolated from abdominal aortic aneurysm tissue and is specifically reactive with T-lymphocytes.

13. A method of detecting collagen I, collagen III, epitope, or combination thereof comprising:
(a) obtaining a sample of aortic tissue;
(b) incubating the sample with one or more T-lymphocytes under suitable conditions so as to react the T-lymphocytes with collagen I, collagen III, epitope or combination thereof and (c) detecting collagen I, collagen III, epitope thereof or combination thereof by measuring proliferation, cytokine release or other reactivity of T-lymphocytes, wherein the response of the T-lymphocytes indicates the presence of collagen I, collagen III, epitope thereof, or combination thereof.


SEQUENCE LISTING (3 sequences)

SEQ ID NO: 1 - protein, 1464 aa, Homo Sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1366 aa, Homo Sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPNGSAGPPGFPGAPGPKGEIGAVGNAGPTGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPDGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAHGAVGAPGPAGATGDRGEAGAAGPAGPAGPRGSPGERGEVGPAGPNGFAGPAGAAGQPGAKGERGGKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGFKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMEAIKVYCDFPTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADHEFFVDIGPVCFK

SEQ ID NO: 3 - protein, 1466 aa, Homo Sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL